(19) United States
(12) Patent Application Publication
(10) Pub. No.: US 2016/0114062 A1
(43) Pub. Date: Apr. 28, 2016

(54) TREATMENT OF IMMUNE, INFLAMMATORY AND DEGENERATIVE ARTHRITIDES WITH TIN-117m

(71) Applicant: RHEUMCO LLC, The Woodlands, TX (US)

(72) Inventors: Gilbert R Gonzales, Tocson, AZ (US); Nigel R. Stevenson, Sugar Hill, GA (US)

(21) Appl. No.: 14/895,222

(22) PCT Filed: Jun. 5, 2014
(86) PCT No.: PCT/US2014/041065

§ 371 Date: Dec. 2, 2015

Related U.S. Application Data
(60) Provisional application No. 61/831,177, filed on Jun. 5, 2013.

(51) Int. Cl. A61K 51/08

(52) U.S. Cl. CPC A61K 51/087

(57) ABSTRACT

Immune, inflammatory and degenerative arthritides, including rheumatoid arthritis, hemophiliac arthropathy and osteoarthritis, can be treated using a systemic or intra-articular radiosynovectomy technique in which the radionuclide is high specific activity tin-117m. Tin-117m can either be bound to a colloid typically used in an intra-articular radiosynovectomy (RSV), or can be tin-117m annexin for RSV and systemic administration.

### RELATED APPLICATION

The present application claims priority to U.S. Ser. No. 61/831,177, filed Jun. 5, 2013, the disclosure of which is hereby incorporated herein by reference in its entirety.

### BACKGROUND OF THE INVENTION

Radiosynovectomy (RSV) is a local intra-articular injection of radionuclides generally in colloidal form for treatment of rheumatoid arthritis and hemophiliac arthritis in man. Evidence in animals also indicate a positive effect (treatment) for degenerative and osteoarthritis by this same method. Typically, RSV employs Y-90, P-32, Re186, Re-188 and Er-169. These all are beta-emitting isotopes with a half life bearing from 2.7 to 14.3 days, and an average penetration of 0.3 to 3.6 millimeters. Beta-emitting isotopes may not be ideal for such treatment, but they are effective in certain applications.

### SUMMARY OF THE INVENTION

The present invention is premised on the realization that immune, inflammatory and degenerative arthritides can be treated by intra-articular injection of high specific activity tin-117m compounds. In particular, tin-117m colloidal compounds can be injected for treatment of rheumatoid arthritis, hemophiliac arthropathy and osteoarthritis.

In a further improvement of the present invention, tin-117 annexin compounds can be injected intra-articularly or systemically for treatment of rheumatoid arthritis, hemophiliac arthropathy and osteoarthritis. The tin-117m accumulates in the affected tissue providing a strong radiation dose from conversion electrons, which travels a very precise distance. The radiation has an effective distance of 290 microns (depending on the density of the tissue traversed), minimizing damaging nearby cells.

The present invention will be further appreciated in light of the following detailed description and drawings in which:

### BRIEF DESCRIPTION OF THE DRAWINGS

FIG. 1 is a graph showing the average ankle measurements of subjects in the second study over time. Ankle size is a measure of the effectiveness of the collagen induced “rheumatoid arthritis-like” model;

FIG. 2 is an autoradiograph showing tin-117m colloid injected in a subject in the first study. In this example, “honeycomb” collimators were inserted between the sample and detector in order to provide superior resolution images;

FIGS. 3 are autoradiographs of test subjects taken and showing tin-117m annexin in test subjects in the second study. In this example, several animal joints were simultaneously viewed;

### DETAILED DESCRIPTION

Tin-117m is injected into the synovium of an affected joint to treat rheumatoid arthritis, hemophiliac arthropathy and osteoarthritis by radiosynovectomy.

Tin-117m annexin is preferably formed from a high specific activity (HSA) no-carrier-added tin-117m (>100 Ci/g). There are various methods to produce no-carrier added tin-117m. One such method is disclosed in Stevenson U.S. Pat. No. 8,257,681, the disclosure of which is hereby incorporated by reference, although other methods of forming the tin-117m could be employed in the present invention. Tin-117-m is a unique radioisotope. Although it emits some gamma radiation, the primary source of the therapeutic radiation is conversion electrons. The radiation from the conversion electron penetrates up to 290 microns, and, therefore, does not damage cells any farther than 290 microns away from the tin atom. This allows for the use of higher doses since the distant radio-sensitive regions such as bone marrow will be spared.

Tin-117m may be bonded to an annexin molecule. Annexins are a class of molecule having the ability to bind with high affinity to membrane lipids in the presence of millimolar concentrations of calcium. There are several different annexins. The term “annexin” includes native annexin purified from natural sources, such as human placenta or annexin molecules containing a native sequence produced through, for example, genetic engineering, or other means. The term annexin unless otherwise specified, includes annexins derived from or produced by any source.

Typically a linking molecule is used to attach the tin-117m annexin. One such linking molecule is 1,4,7,10tetraazacylododecane-1,4,7,10-tetra acetic acid, or DOTA. Usually, aminobenzyl DOTA is used. Formation of this molecule is disclosed in U.S. Pat. No. 8,283,167, the disclosure of which is hereby incorporated by reference.

According to the present invention, the tin-117m annexin V in a suitable sterile carrier, such as saline, is injected directly into the affected joint or is systemically injected to treat multiple joints. Annexin V is known to bind to phosphatidyl serine which is exposed in cells during apoptosis. Recent work in the imaging and treatment of vulnerable or unstable plaque provided strong evidence that very low doses of tin-117m annexin were able to have positive effects. The data supports the observation that the tin-117m annexin is inducing apoptosis even at these “hormesis” doses which allows for treating various arthritides with low quantities of tin-117m. The dosage may vary widely depending on the particular joint, as well as the extent of the damage. Generally, 0.1 milliCuries to 20 milliCuries will be injected, or, more particularly, 0.1 milliCuries to 5 milliCuries. According to one embodiment of the present invention, a dosage of HSA tin-117m is sufficient to provide conversion electron therapy which is hormetic. A much lower dosage provides hormesis. Generally, 0.01 to <0.1 milliCuries can be typically used for hormesis doses. These injections may be repeated after a period of time, and, further, gamma emissions from the injected tin-117m can be imaged, if desired.

According to a second aspect of the present invention, a tin-117m colloidal solution is injected into an affected joint, again, in an RSV procedure. Any colloid typically used for RSV purposes can be used in the present invention. Typical particles include citrate/silicate; colloid/sulfide, hydroxide and citrate. These are injected intra-articularly in the same manner as the tin-117m annexin V, and, further, in similar dosage. Typical particle sizes range from 0.1-20 μm, or more particularly, 3-20 μm.

Of particular note in this invention is the ability to use known cold (non-radioactive) tin colloids that are subsequently coated with HSA Sn-117m. This lends itself to a kit preparation in which a radiopharmacy would maintain colloidal cold tin. The HSA tin-117m would be sent to the pharmacy where it would be mixed with the cold tin and provided to the doctor or veterinarian for injection.

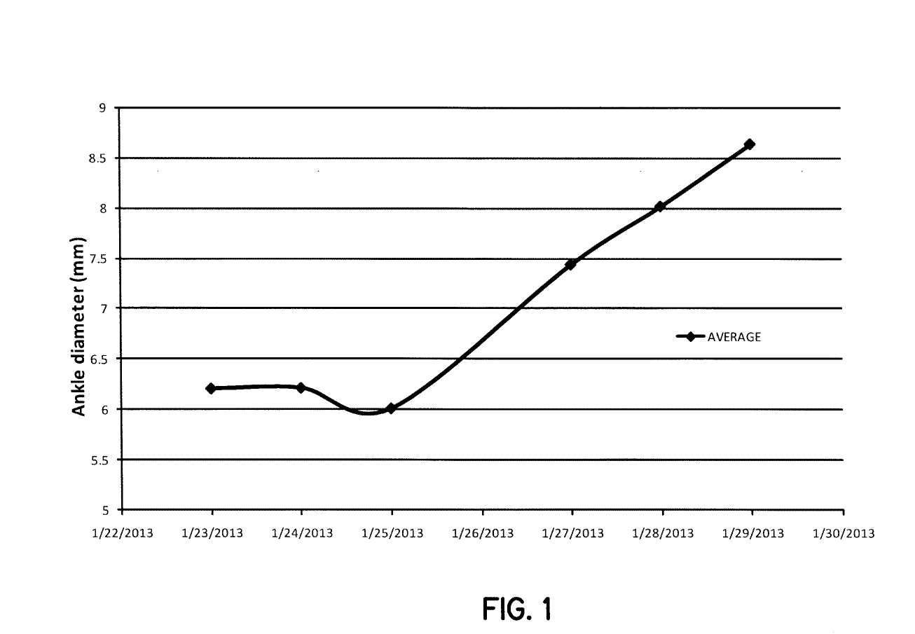

In order to test the efficacy of the present invention, a standard arthritic rat model was employed. Specifically, rodents were injected with collagen, which induced a reaction similar to rheumatoid arthritis. FIG. 1 shows how the model is validated by measurements of ankle size that increase after collagen injections. Two different studies were conducted. Table 1 shows the experiment design for the first study.

TABLE 1
Experimental Design for HSA Sn-117m Colloid Study
Experimental Design - Study 1
Schedule for Inducing Arthritis and Dosing with Sn-117m COLLOID

| | Day 0 | Day 1 | Day 2 | Day 3 | Day 4 | Day 5 | Day 6 | Day 7 | Day 8 |
|---|---|---|---|---|---|---|---|---|---|
| | 11-Dec | 12-Dec | 13-Dec | 14-Dec | 15-Dec | 16-Dec | l7-Dec | 18-Dec | 19-Dec |
| Rat 1 | a | | | | | | | b w | + |
| Rat 2 | a | | | | | | | b w | + |
| Rat 3 | a | | | | | | | b w | + |
| Rat 4 | a | | | | | | | b w | + |
| Rat 5 | a | | | | | | | b y | + |
| Rat 6 | a | | | | | | | b y | + |

| | Day 9 | Day 10 | Day 11 | Day 12 | Day 13 | Day 14 | Day 15 |
|---|---|---|---|---|---|---|---|
| | 20-Dec | 21-Dec | 22-Dec | 23-Dec | 24-Dec | 25-Dec | 26-Dec |
| Rat 1 | + | + | + | + | + | + | q + |
| Rat 2 | + | + | + | + | + | + | q + |
| Rat 3 | x + | + | + | + | + | + | q + |
| Rat 4 | x + | + | + | + | + | + | q + |
| Rat 5 | z + | + | + | + | + | + | q + |
| Rat 6 | + | + | + | + | + | + | q + |

a - Inject each rat with 200 uL of collagen on the left and 200 uL on the right side.
b - Booster injection 100 uL of collagen between the two previous injection sites.
w - Dose with ~8.3 uCi Sn-117m colloid
x - Dose with ~33.2 uCi Sn-117m colloid
y - Dose with ~8.3 uCi Sn-117m Homogenous Precipitation
z - Dose with ~33.2 uCi Sn-117m Homogenous Precipitation
+ - Measure ankles with calipers and record the information.
q - Sacrifice and perform full biodistribution including removing thymus, feet and legs. Place thymus, liver, spleen, feet and legs into 10% formalin

200 micro liters of collagen were injected on the left and right knees at day zero. At day seven, booster injections of 100 micro liters of collagen were again injected and also 8.3 μCi of tin-117m colloid. Two days later an additional 33.2 μCi of tin-117m colloid was added to half of the animals for a total dose of 41.5 μCi.

TABLE 2
Experimental Design for HSA Sn-117m Annexin Study
Experimental Design - Study 2
Schedule for Inducing Arthritis and Dosing with Sn-117m ANNEXIN

| | Day 0 | Day 1 | Day 2 | Day 3 | Day 4 | Day 3 | Day 6 | Day 7 |
|---|---|---|---|---|---|---|---|---|
| | 15-Jan | 16-Jan | 17-Jan | 18-Jan | 19-Jan | 20-Jan | 21-Jan | 22-Jan |
| Rat 1 | a | | | | | | | b |
| Rat 2 | a | | | | | | | b |
| Rat 3 | a | | | | | | | b |
| Rat 4 | a | | | | | | | b |

| | Day 8 | Day 9 | Day 11 | Day 11 | Day 12 | Day 13 | Day 14 |
|---|---|---|---|---|---|---|---|
| | 23-Jan | 24-Jan | 25-Jan | 26-Jan | 27-Jan | 28-Jan | 29-Jan |
| Rat 1 | + | + | x + | + | + | + | z + |
| Rat 2 | + | + | x + | + | + | + | z + |
| Rat 3 | + | + | y + | + | + | + | z + |
| Rat 4 | + | + | y + | + | + | + | z + |

a - Inject each rat with 200 uL of collagen on the left and 200 uL on the right side.
b - Booster injection 100 uL of collagen between the two previous injection sites.
x - Dose with ~50 uCi Sn-117m Annexin (retro-orbital)
y - Dose with ~50 uCi Sn-117m Annexin (Synovium)
+ - Measure ankles with calipers and record the information
z - Sacrifice and perform full biodistribution including removing thymus, feet and legs. Place fet and legs into 10% formalin.

Table 2 shows the similar rat study, but 50 μCi of tin-117m-DOTA annexin was either directly injected into the synovium, or retro-orbitally to determine systemic efficacy. At around day 14, the animals were sacrificed and tissues sent for histological analyses.

TABLE 3
Biodistribution Results for HSA Sn-117m Colloid Study
1^{st} Study - Biodistribution

| | | Dose (mCi) | | Right Leg |
|---|---|---|---|---|
| Animal | Injection Location | (St/Ho) | Left leg (% ID) | (% ID) |
| 1 | L Synovium | 8.3 (St) | 65.9 | 2.3 |
| 2 | L Synovium | 8.3 (St) | 62.8 | 2.3 |
| 3 | L Synovium | 41.5 (St) | 68.2 | 2.0 |
| 4 | L Synovium | 41.5 (St) | 68.7 | 2.0 |
| 5 | L Synovium | 41.5 (Ho) | 98.7 | 0.0 |
| 6 | L Synovium | 8.3 (Ho) | 99.9 | 0.0 |

TABLE 4
Biodistribution Results for HSA Sn-117m Annexin Study
2^{nd} Study - Biodistribution

| | | Dose | Left leg | Right Leg |
|---|---|---|---|---|
| Animal | Injection Location | (mCi) | (% ID) | (% ID) |
| 1 | Retro-orbital | 50 | 0.1 | 0.1 |
| 2 | Retro-orbital | 50 | 0.1 | 0.1 |
| 3 | L Synovium | 50 | 20.1 | 0.1 |
| 4 | L Synovium | 50 | 7.3 | 0.1 |

TABLE 5
Histopathology Results for HSA Sn-117m Colloid Study
Histopathology - 1^{st} Study (Knees)

| Treatment | | Species: Rat | | | | |
|---|---|---|---|---|---|---|
| Group | | Necropsy Day: X | | | | |
| Group 1 | | Histopathology Score; (0-5, 0 = normal, 5 = severe) | | | | Summed |
| N/A | | Inflammation | Pannus | Cartilage Damage | Bone Resorption | Histopathology |
| 0 | | Score | Score | Score | Score | Scores |
| 1 | R | 4 | 0.5 | 0.5 | 0 | 5 |
| | L | 4 | 0.5 | 1 | 0.5 | 6 |
| 2 | R | 2 | 0.5 | 1 | 0 | 3.5 |
| | L | 5 | 1 | 1 | 1 | 8 |
| 3 | R | 2 | 0.5 | 1 | 0 | 3.5 |
| | L | 4 | 1 | 1 | 0 | 6 |
| 4 | R | 4 | 1 | 1 | 1 | 7 |
| | L | 4 | 1 | 1 | 0.5 | 6.5 |
| 5 | R | 5 | 2 | 2 | 2 | 11 |
| | L | 5 | 1 | 2 | 1 | 9 |
| 6 | R | 5 | 1 | 1 | 1 | 8 |
| | L | 5 | 1 | 2 | 1 | 9 |

| | | | | | |
|---|---|---|---|---|---|
| Mean | 4.08 | 0.92 | 1.21 | 0.67 | 6.88 |
| SE | 0.31 | 0.12 | 0.14 | 0.18 | 0.65 |
| ANOVA Results (grp 2) | #REF! | #REF! | #REF! | #REF! | #REF! |
| % Inhibition to G2 | #REF! | #REF! | #REF! | #REF! | #REF! |

TABLE 6
Histopathology Results for HSA Sn-117m Annexin Study
Histopathology - 2^{nd} Study (Knees)

| Treatment | | Species: Rat | | | | |
|---|---|---|---|---|---|---|
| Group | | Necropsy Day: X | | | | |
| Group 1 | | Histopathology Scores (0-5, 0 = normal, 5 = severe) | | | | Summed |
| 0 | | Inflammation | Pannus | Cartilage Damage | Bone Resorption | Histopathology |
| 0 | | Score | Score | Score | Score | Scores |
| 1 | R | 0.5 | 0.5 | 0.5 | 0 | 1.5 |
| | L | 5 | 1 | 1 | 1 | 8 |
| 2 | R | 4 | 0.5 | 0.5 | 0.5 | 5.5 |
| | L | 3 | 0.5 | 0.5 | 0 | 4 |
| 3 | R | 5 | 1 | 2 | 1 | 9 |
| | L | 5 | 1 | 2 | 1 | 9 |
| 4 | R | 5 | 1 | 1 | 0.5 | 7.5 |
| | L | 5 | 1 | 2 | 1 | 9 |

| | | | | | |
|---|---|---|---|---|---|
| Mean | 4.04 | 0.92 | 1.33 | 0.75 | 7.04 |
| SE | 0.48 | 0.12 | 0.21 | 0.17 | 0.89 |

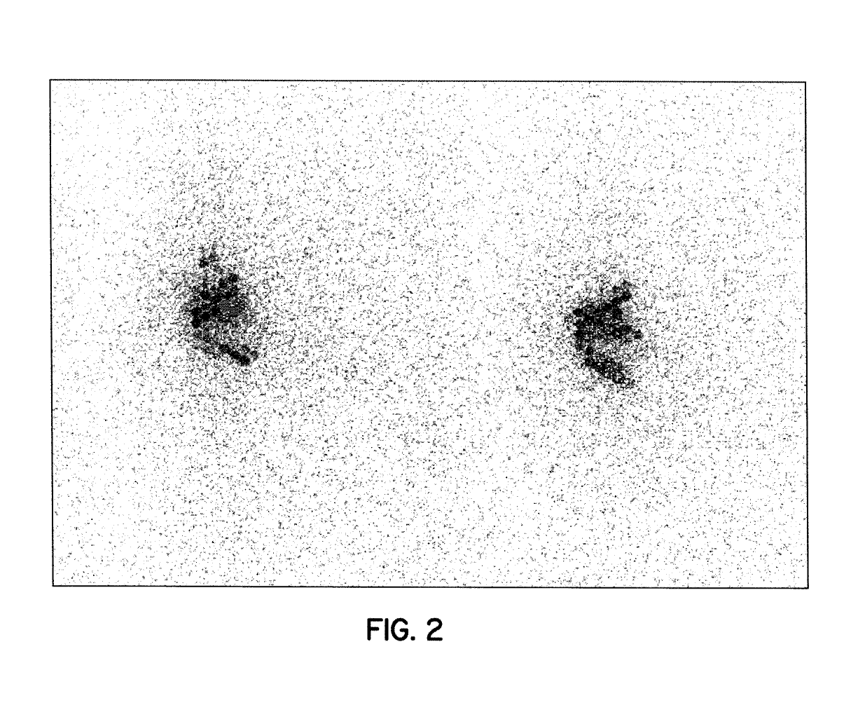

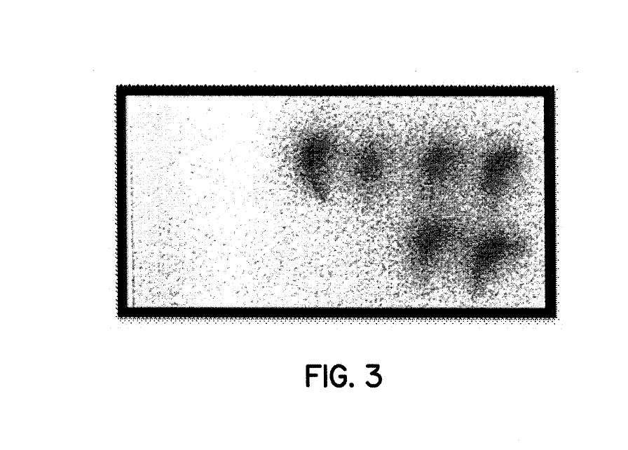

Tables 3 and 4 show the biodistribution of the radioactive product at the end of the studies. This varies significantly depending on the colloid formulation or the mode of administration of the annexin product. However, very high retention is observed for some colloids in particular. FIGS. 2 and 3 show radiographs of the localized containment of the intra-articular injections for both studies.

Tables 5 and 6 show the histopathology results for the two studies. In the first study, synovial colloidal suspension injections of tin-117m appears to be effective and the product is uniformly distributed throughout the synovial joint cavity. Likewise, data from the second study suggests that the tin-117-DOTA annexin appears to be effective when administered directly into the synovium. The study does serve to prove the validity of the model for this application and suggests the therapeutic effectiveness of Sn-117m.

This has been a description of the present invention along with the preferred method of practicing the present invention. However, the invention itself should only be defined by the appended claims, WHEREIN WE CLAIM:

What is claimed is:

1. A method of treating immune, inflammatory and degenerative arthritides such as rheumatoid arthritis, hemophiliac arthropathy and osteoarthritis comprising injecting high specific activity tin-117m intra-articularly into a joint or systemically in humans or animals.

2. The method claimed in claim 1 wherein said tin-117m is a high specific activity tin-117m colloidal compound that is injected intra-articularly.

3. The method claimed in claim 2 wherein said colloidal compound is a non-radioactive tin colloid base that is coated with radioactive high specific activity Sn-117m.

4. The method claimed in claim 1 wherein said tin-117m is high specific activity tin-117m annexin which is injected intra-articularly.

5. The method claimed in claim 4 wherein said annexin is annexin V.

6. The method claimed in claim 1 wherein 0.1 to 20 milliCuries of high specific activity tin-117m are injected in each joint.

7. The method claimed in claim 1 wherein 0.01 to <0.1 milliCuries of high specific activity tin-117m are injected in each joint providing hormetic therapy.

8. The method claimed in claim 5 wherein 1 to 40 milliCuries of high specific activity tin-117m are injected systemically.

9. The method claimed in claim 1 further comprising combining high specific activity tin-117m with non-radioactive colloidal tin to form tin-117m coated colloidal tin and injecting said coated colloidal tin intra-articularly.
